(19) United States
(12) Patent Application Publication
(10) Pub. No.: US 2009/0281564 A1
(43) Pub. Date: Nov. 12, 2009

(54) Pre-Clot Vessel Dilator

(75) Inventor: Daphne A. Kontos, Washington, DC (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 12/118,035

(22) Filed: May 9, 2008

(51) Int. Cl. A61M 29/02

(52) U.S. Cl. 606/194

(57) ABSTRACT

A pre-clot dilator is described that may be used to dilate hardened regions of an occluded region. The pre-clot is provided with one or more struts that extend along a surface of a balloon. Each strut has a proximal fixed end and a distal free end. The distal free end extends past the distal end of the shaft. The distal free end is designed with a penetrating tip that is capable of boring through a proximal portion of the occlusion. Expansion of the balloon allows the strut to pivot about the proximal fixed end of the strut, thereby producing a force sufficient to push out the occlusion towards the vessel wall. The balloon may be deflated so that the strut can collapse over the balloon to create a low profile which enables distal advancement deeper into the occlusion. The procedure is repeated until the occlusion is separated.

### BACKGROUND

The present invention relates generally to medical devices and more particularly to balloon catheters used to dilate narrowed portions of a lumen.

Balloon catheters are widely used in the medical profession for various intraluminal procedures. One common procedure involving the use of a balloon catheter relates to angioplasty dilation of coronary or other arteries suffering from stenosis (i.e., a narrowing of the arterial lumen that restricts blood flow).

Although balloon catheters are used in many other procedures as well, coronary angioplasty using a balloon catheter has drawn particular attention from the medical community because of the growing number of people suffering from heart problems associated with stenosis. This has lead to an increased demand for medical procedures to treat such problems. The widespread frequency of heart problems may be due to a number of societal changes, including the tendency of people to exercise less while eating greater quantities of unhealthy foods, in conjunction with the fact that people generally now have longer life spans than previous generations. Angioplasty procedures have become a popular alternative for treating coronary stenosis because angioplasty procedures are considerably less invasive than other alternatives. For example, stenosis of the coronary arteries has traditionally been treated with bypass surgery. In general, bypass surgery involves splitting the chest bone to open the chest cavity and grafting a replacement vessel onto the heart to bypass the blocked, or stenosed, artery. However, coronary bypass surgery is a very invasive procedure that is risky and requires a long recovery time for the patient.

To address the increased need for coronary artery treatments, the medical community has turned to angioplasty procedures, in combination with stenting procedures, to avoid the problems associated with traditional bypass surgery. Typically, angioplasty procedures are performed using a balloon-tipped catheter that may or may not have a stent mounted on the balloon (also referred to as a stented catheter). The physician performs the angioplasty procedure by introducing the balloon catheter into a peripheral artery (commonly one of the leg arteries) and threading the catheter to the narrowed part of the coronary artery to be treated. During this stage, the balloon is uninflated and collapsed onto the shaft of the catheter in order to present a low profile which may be passed through the arterial lumens. Once the balloon is positioned at the narrowed part of the artery, the balloon is expanded by pumping a mixture of saline and contrast solution through the catheter to the balloon. As a result, the balloon presses against the inner wall of the artery to dilate it. If a stent is mounted on the balloon, the balloon inflation also serves to expand the stent and implant it within the artery. After the artery is dilated, the balloon is deflated so that it once again collapses onto the shaft of the catheter. The balloon-tipped catheter is then retracted from the arteries. If a stent is mounted on the balloon of the catheter, the stent is left permanently implanted in its expanded state at the desired location in the artery to provide a support structure that prevents the artery from collapsing back to its pre-dilated condition. On the other hand, if the balloon catheter is not adapted for delivery of a stent, either a balloon-expandable stent or a self-expandable stent may be implanted in the dilated region in a follow-up procedure. Although the treatment of stenosed coronary arteries is one common example where balloon catheters have been used, this is only one example of how balloon catheters may be used and many other uses are also possible.

One problem that may be encountered with conventional angioplasty techniques is the proper dilation of stenosed regions that are hardened and/or have become calcified. Stenosed regions may become hardened for a variety of reasons, such as the buildup of atherosclerotic plaque or other substances. Hardened regions of stenosis can be difficult to completely dilate using conventional balloons because hardened regions tend to resist the expansion pressures applied by conventional balloon catheters. Furthermore, the stenosed regions may become fully occluded to the extent that the entire lumen of the vessel is blocked, thereby preventing a dilation device from being deployed within the stenosed region. Although the inventions described below may be useful in treating hardened regions of stenosis, the claimed inventions may also solve other problems as well.

### SUMMARY

The invention may include any of the following aspects in various combinations and may also include any other aspect described below in the written description or in the attached drawings.

In a first aspect, a pre-clot dilator is provided comprising a balloon having a distal portion, and a proximal portion, wherein at least a length of an outer surface of the balloon comprises a working diameter; a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state; and at least one strut comprising a distal free end disposed past the distal end of the shaft, the strut proximally extending from the distal free end along the outer surface of the balloon and terminating at a proximal fixed end, the distal free end comprising a penetrating tip configured to pierce into an occlusion and upon expansion of the balloon move from a retracted state to an extended state away from the longitudinal axis of the shaft to spread apart the occlusion.

In a second aspect, a pre-dilator is provided comprising a balloon having a distal portion, and a proximal portion, wherein at least a length of an outer surface of the balloon comprises a working diameter; a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state; a first strut comprising a first distal free end disposed past the distal end of the shaft, the first strut proximally extending from the from the first distal free end along the outer surface of the balloon and terminating at a first proximal fixed end attached to the distal end of the shaft; a second strut spaced apart from the first strut, the second strut comprising a second distal free end disposed past the distal end of the shaft, the second strut proximally extending from the from the second distal free end along the outer surface of the balloon and terminating at a second proximal fixed end attached to the distal end of the shaft; and wherein the first distal free end comprises a first penetrating tip and the second distal free end comprises a second penetrating tip, the first penetrating and the second penetrating tips pivotable from a retracted position to an extended state away from the longitudinal axis of the shaft.

In a third aspect, a method for dilating an occluded region, comprising the steps of: (a) providing a pre-clot dilator having a balloon having a distal portion, and a proximal portion, wherein at least a length of an outer surface of the balloon comprises a working diameter; a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state; at least one strut comprising a distal free end disposed past the distal end of the shaft, the strut proximally extending from the distal free end along the outer surface of the balloon and terminating at a proximal fixed end, the distal free end comprising a penetrating tip; (b) advancing the pre-clot dilator proximal to the occluded region with the at least one strut collapsed along the outer surface of the balloon; (c) piercing the occlusion with the penetrating tip; (d) expanding the balloon to the inflated state; and (e) extending the at least one strut outward as the balloon is expanding to the inflated state to spread apart the occlusion.

### BRIEF DESCRIPTION OF THE DRAWINGS

The invention may be more fully understood by reading the following description in conjunction with the drawings, in which:

FIG. 1 is a side view of a pre-clot dilator including a first strut and a second strut shown in their retracted state about the outer surface of the balloon;

FIG. 2 shows the pre-clot dilator of FIG. 1 with the struts extended outward;

FIG. 3 shows a blown-up view of a distal free end of a strut having barbs disposed therealong the surface;

FIG. 4 shows a blown-up view of a distal free end of a strut having barbs extending away from a surface of the distal free end;

FIG. 5 shows another example of the distal free end comprising multiple anchor or barb elements along its outer surface;

FIG. 6 shows a blown up view of the distal free ends;

FIG. 7 shows an elastic band for ensuring that the struts return to their retracted state and are situated in close proximity to the outer surface of the balloon;

FIG. 8 shows a proximal end of the pre-clot dilator with an aspirating element attached to a proximal end of a lumen of the shaft; and

FIGS. 9-12 show a procedure for using the pre-clot dilator to push out an occluded region.

### DETAILED DESCRIPTION

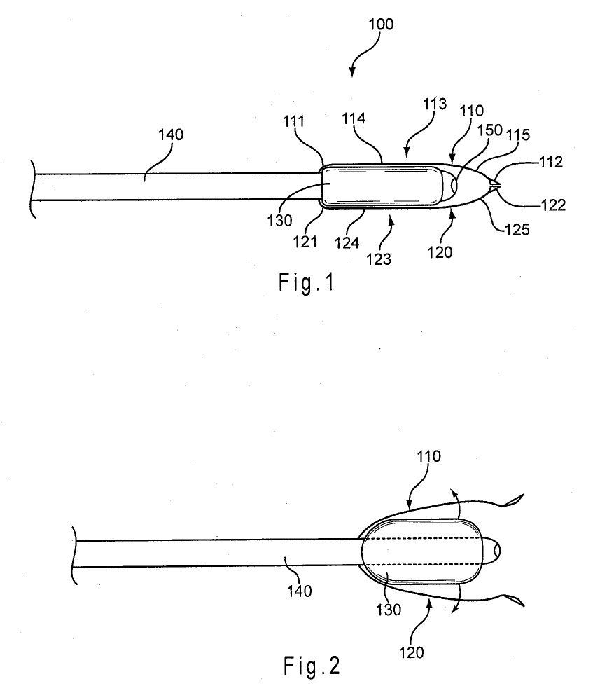

FIGS. 1-3 show a pre-clot dilator 100 used for spreading apart an occluded region within a vessel wall. Generally speaking, the pre-clot dilator 100 may be used to navigate through hard-to-cross lesions (i.e., clots) which are occluded to the extent that a guide wire cannot pass therethrough. As will be explained in greater detail below, the pre-clot dilator 100 may be used to separate apart the lesion and create a gap sufficient for a guide wire or other medical device to pass therethrough. The pre-clot dilator 100 advances adjacent and proximal to the lesion. The distal ends of one or more struts extending along the balloon grab and pierce the lesion and thereafter radially extend outward toward the vessel wall as the balloon expands to its inflated state. This mechanism separates the occluded region of the vessel.

FIG. 1 shows a distal end of the pre-clot dilator 100 comprising a first strut 110 and a second strut 120. The struts 110 and 120 are shown collapsed over the surface of a deflated balloon 130. When the balloon 130 expands into an inflated state as shown in FIG. 2, the struts 110 and 120 extend out away from a longitudinal axis of the shaft 140 to allow separation of an occluded region within a vessel wall, thereby enabling a device such as a guide wire to gain access through the occluded region and to a target stenosed site where angioplasty can occur. Such a procedure will be discussed in greater detail below.

Referring to FIG. 1, the first strut 110 comprises a proximal fixed end 111, a distal free end 112, and a body portion 113. The proximal fixed end 111 is shown preferably fixedly attached to an outer surface the shaft 140 along the distal end of the shaft 140. The proximal fixed end 111 may be attached to the outer surface of the shaft 140 by an adhesive, spot weld, or other means known to one of ordinary skill in the art. Alternatively, the proximal fixed end 111 may be fixedly attached to an outer surface of the balloon 130. The body portion 113 of the first strut 110 comprises a first portion 114 which extends along the outer surface of the balloon 130. The first portion 114 is not attached to the outer surface of the balloon 130 so that it can extend radially outwards and proximally relative to the balloon 130 towards the vessel wall as the balloon 130 undergoes expansion to an inflated state. In other words, the first portion 114 of the body portion 113 pivots about proximal fixed end 111 in an arc-like movement during expansion of the balloon 130. The body portion 113 also comprises a second portion 115 which extends past the distal end 150 of shaft 140. The second portion 115 preferably tapers inwards towards the longitudinal axis of the shaft, thereby creating a lower profile of the pre-clot dilator 100 during delivery to the occluded region. The second portion 115 also pivots in an arc-like movement during expansion of the balloon 130.

The second strut 120 also comprises similar components to the first strut 110: a proximal fixed end 121, a distal free end 122, and a body portion 123. The second strut 120 is shown circumferentially spaced apart from the first strut 110. The exact spaced apart distance may vary depending on the geometry and type of occluded region as well as the specific vessel the pre-clot dilator is being used within. Still referring to FIG. 1, the proximal fixed end 121 is shown preferably fixedly attached to an outer surface the shaft 140 along the distal end of the shaft 140. The proximal fixed end 121 may be attached to the outer surface of the shaft 140 by an adhesive, spot weld, or other means known to one of ordinary skill in the art. Alternatively, the proximal fixed end 121 may be fixedly attached to an outer surface of the balloon 130. The body portion 123 of the second strut 120 comprises a first portion 124 which extends along the outer surface of the balloon 130. The first portion 124 is not attached to the outer surface of the balloon 130 so that it can extend radially outwards and proximally relative to the balloon 130 towards the vessel wall as the balloon 130 undergoes expansion to an inflated state. In other words, the first portion 124 pivots about proximal fixed end 121 in an arc-like movement during expansion of the balloon 130. The body portion 123 also comprises a second portion 125 which extends past the distal end 150 of shaft 140. The second portion 125 preferably tapers inwards towards the longitudinal axis of the shaft 140, thereby creating a lower profile of pre-clot dilator 100 during delivery to the occluded region. The second portion 125 also pivots in an arc-like movement during expansion of the balloon 130.

The first portions 114 and 124 are shown in FIG. 1 to be substantially parallel to a longitudinal axis of the shaft 140 when struts 110 and 120 are in their retracted state. However, other configurations of first portions 114 and 124 are contemplated. For example, the first portions 114 and 124 may be oriented non-parallel to each other about the outer surface of the balloon 130.

The longitudinal length of the struts 110 and 120 are preferably greater than the longitudinal length of the balloon 130 to allow engagement and subsequent separation of the occlusion, as will become apparent during description of the method of use of pre-clot dilator 100.

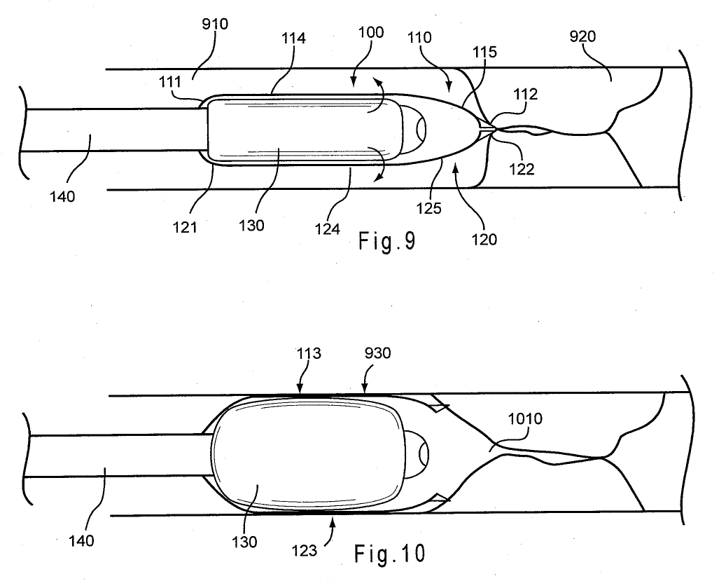

The distal free end 112 of the first strut 110 and the distal free end 122 of the second strut 120 preferably comprise penetrating tips, which may be sharpened edges to enable engagement of the occlusion. The penetrating tips of distal free end 112 and distal free end 122 may be sufficiently sharp to penetrate into a predetermined proximal portion of the calcified lesion. The distal free end 112 is substantially parallel to the distal free end 122, as shown in blown up view FIG. 6. After penetrating and securing itself within the proximal portion of the calcified lesion, the balloon 130 is expanded to an inflated state. Body portion 113 of strut 110 radially extends outward in a first direction and body portion 123 of strut 120 radially extends outward in a second direction to separate the occluded region into two lesions with a gap extending therethrough (as shown in FIGS. 9-12). The gap is sufficiently sized to allow a guide wire to extend therethrough. The first direction is preferably opposite to the second direction. However, the struts 110 and 120 may be disposed about the balloon 130 such that the first direction and the second direction may comprise any clockwise or counterclockwise angular direction. The precise type of spacing may be dependent upon numerous factors, including the geometry of the occlusion and the vessel.

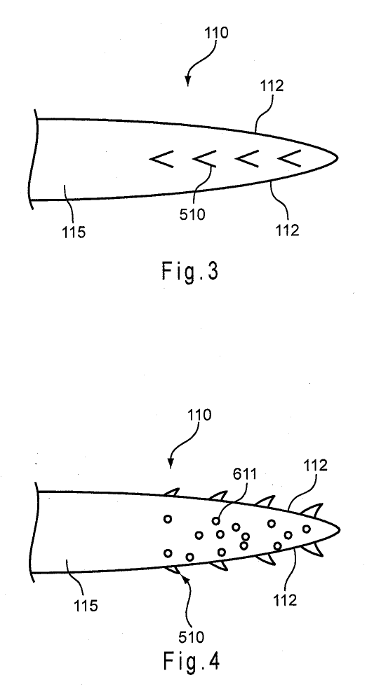

Various configurations of the penetrating tips are contemplated. For example, FIG. 3 shows that the outer surface of distal free end 112 may be covered with barbs 510 that can be formed along the surface of distal free end 112. The barbs 510 provide a sandpaper effect of raised, pointed, directional bumps along the surface of the distal free end 112. The barbs 510 may also be non-directional bumps which are oriented in a random fashion along the surface of distal free end 112. Alternatively, FIG. 4 shows another embodiment in which multiple barbs 510 are formed along the outer surface of distal free end 112 so that the barbs 510 are directed outwardly from the surface. The barbs 510 are shown oriented substantially perpendicular to the outer surface of distal free end 112. The orientation of barbs 510 as shown in FIG. 3 or FIG. 4 enables sufficient gripping of the vessel wall and the proximal portion of the occlusion after penetrating into the occlusion, thereby preventing the likelihood of slippage from the occlusion site during the dilation procedure.

Still referring to FIG. 4, the penetrating tips of distal free ends 112 and 122 may also comprise a textured surface to provide frictional engagement with the lesion. Surface indentations may be utilized to create a textured surface. The surface indentations may be dimples or grooves 611 that extend along the outer surface of the strut 110 to create the textured surface, as shown in FIG. 4. A variety of different shaped surface indentations are contemplated, including spherical, elliptical, rectanguloid. A variety of different sized surface indentations are also contemplated. Furthermore, surface protrusions are also contemplated, such as thin ribbed surfaces.

Any number of barbs 510 as well as their orientation along the surface of the distal free end 112 is contemplated. At least one of the barbs 510 shown in FIGS. 3 and 4 is pierced into the proximal portion of the lesion. Preferably all of the barbs 510 extending along the outer surface of the distal free end 112 are penetrated into the proximal portion of the lesion to enhance engagement of the distal free end 112 within the lesion. Accordingly, the distal free ends 112 and 122 should comprise a sufficient longitudinal length so that a sufficient number of barbs 510 can be disposed along the outer surface of the distal free ends 112 and 122 of first and second struts 110 and 120.

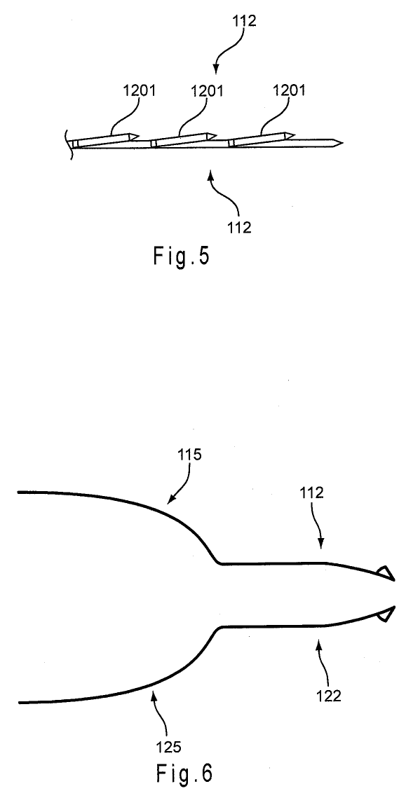

Additionally, FIG. 5 shows another example of distal free end 112 comprising multiple anchor or barb elements along its outer surface. In particular, the surfaces of distal free end 112 are shown covered with barbs 1201. FIG. 5 shows that the barbs 1201 angle outwardly a predetermined amount from the surface of distal free end 112. The barbs 1201 provide a sandpaper effect of raised, pointed, directional bumps extending away from the surface of the distal free end 112. Each of the barbs 1201 preferably faces in substantial alignment with a common longitudinal axis of distal free end 112.

Combinations of the above-described surface features are contemplated. For example, the outer surfaces of the struts 110 and 120 may comprise dimples as well as barbs or other anchoring elements It should be recognized that the above-described penetrating tips of distal free ends 112 and 122 can be provided in a variety of shapes and configurations other than shown to insure adequate penetration and engagement within the lesion.

Because only the distal free ends 112 and 122 are designed to penetrate into the clot, the outer surfaces of the body portions 113 and 123 are preferably smooth and atraumatic to prevent potential cutting and damaging of normal, healthy tissue during the procedure. The cross-sectional shape of the body portions 113 and 123 may be characterized by the absence of any type of sharpened edge. Examples of cross-sectional shapes include but are not limited to rounded and tear-dropped.

Expansion of the balloon 130 causes body portions 113 and 123 and respective distal free ends 112 and 122 to pivot about their respective proximal fixed ends 111 and 121 so as to radially extend outwards towards the vessel wall, as shown by the arrows in FIG. 2. FIG. 2 shows that body portions 113 and 123 and respective distal free ends 112 and 122 preferably extend in the radial direction past the inflated balloon 130. Such a degree of radial extension of the body portions 113 and 123 and free ends 112 and 122 may allow sufficient separation of the occluded region. However, body portions 113 and 123 and respective distal free ends 112 and 122 need not pivotally extend radially outward to the extent shown in FIG. 2 to sufficiently separate an occluded region. Generally speaking, the specific extension of the struts 110 and 120 is application specific and can be adjusted by controlling the amount of expansion that the balloon 130 undergoes during the dilation procedure.

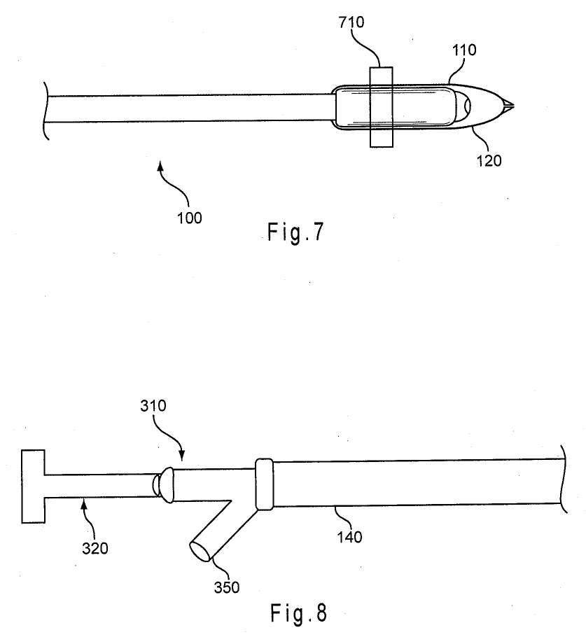

Creating a pre-clot dilator 100 with a sufficiently low profile also is partially dependent upon the ability of the struts 110 and 120 to return to their retracted state in which they are collapsed over the outer surface of the balloon 130. An inability of the struts 110 and 120 to return to their collapsed configuration over the balloon 130 may create an undesirably large profile that may not be easily removable from a patient. Accordingly, FIG. 7 shows a means for ensuring that the struts 110 and 120 return to their retracted state and are situated in close proximity to the outer surface of the balloon 130. FIG. 7 shows a restraining element 710 circumferentially disposed about the balloon 130 and first portions 114 and 124 (see FIG. 1) of body portions 113 and 123. The restraining element 710 is preferably an elastic band which naturally constrains the struts 110 and 120 against an outer surface of the balloon 130 when balloon 130 is deflated. The elasticity of the band 710 allows it to expand with expansion of the balloon 130. As a result, removal of the restraining element 710 may not be required during separation of the occlusion.

Occasionally, some of the plaque particles may loosen from the lesion after separation of an occlusion, accumulate within the vessel and potentially block blood flow. Accordingly, an aspirating element 320 (FIG. 8) may be attached to the proximal end 310 of the pre-clot dilator 100. The aspirating element 320 may be a syringe used to suction particles from the occluded region.

The struts 110 and 120 may be made from any suitable biocompatible material. Such materials may include superelastic materials (e.g., nitinol) and metallic alloys (e.g., stainless steel). Additionally, the struts 110 and 120 may also be formed from polymeric materials such as high density polypropylene.

Any number of struts is contemplated. Although the embodiments show two struts, less than two struts or greater than two struts may be used. The exact number of struts to use may be dependent at least partially on the size of the occluded vessel, the geometry of the occlusion around a vessel wall, and the severity of the occlusion.

Having described the structure of the pre-clot dilator 100, a method of using the pre-clot dilator 100 will be described referring to FIGS. 9-12. The pre-clot dilator 100 may typically be used after a physician is unable to navigate a guide wire or other medical device past an occluded region of a vessel. At this juncture, the physician may use the pre-clot dilator 100 to separate the occlusion, as will now be described. FIG. 9 shows that the pre-clot dilator 100 is navigated along a vessel lumen 910. During navigation, the struts 110 and 120 are in their retracted state in which first portions of body portions 114 and 124 are collapsed onto the deflated balloon 130. The second portions 115 and 125 of body portions 114 and 124 taper inwards, as shown in FIG. 9, such that distal free ends 112 and 122 are in close proximity to each other or contacting each other. The pre-clot dilator 100 is navigated with the struts 110 and 120 in their retracted state to create a low profile. Advancement of the dilator 100 continues until the distal free ends 110 and 122 abut against the proximal portion of the occluded region 920. The distal free ends 112 and 122 comprise penetrating tips which function to pierce into the proximal portion of the occlusion 920, as shown in FIG. 9. The penetrating tips are designed in any of the ways described above so that they pierce through the occlusion 920 and remain engaged with the occlusion 920.

With the distal free ends 112 and 122 penetrated into the proximal portion of the occlusion 920, the balloon 130 is expanded by injecting inflation fluid (e.g., saline solution) through proximal end lumen 350 (FIG. 8) of shaft 140. As the balloon 130 expands, body portions 113 and 123 of struts 110 and 120 pivotally radially extend away from the longitudinal axis of the shaft 140, as shown in FIG. 10. Body portion 113 of strut 110 pivotally extends radially outward in a first direction and body portion 123 of strut 120 pivotally extends radially outward in a second direction substantially opposite to the first direction, as shown by the arrows in FIG. 9. The movement of the struts 110 and 120 in their respective directions allows distal free ends 112 and 122 to separate a proximal portion of the occlusion 920 and create a gap 1010 (FIG. 10) within the occlusion 920. Expansion of the balloon 130 continues until the body portions 113 and 123 contact vessel wall 930. Because the body portions 113 and 123 are preferably designed with unsharpened edges, the tissue along vessel wall 930 is not damaged as body portion 113 of strut 110 and body portion 123 of strut 120 press against the vessel wall 930. The body portions 113 and 123 extend beyond the inflated balloon 130, as shown in FIG. 10.

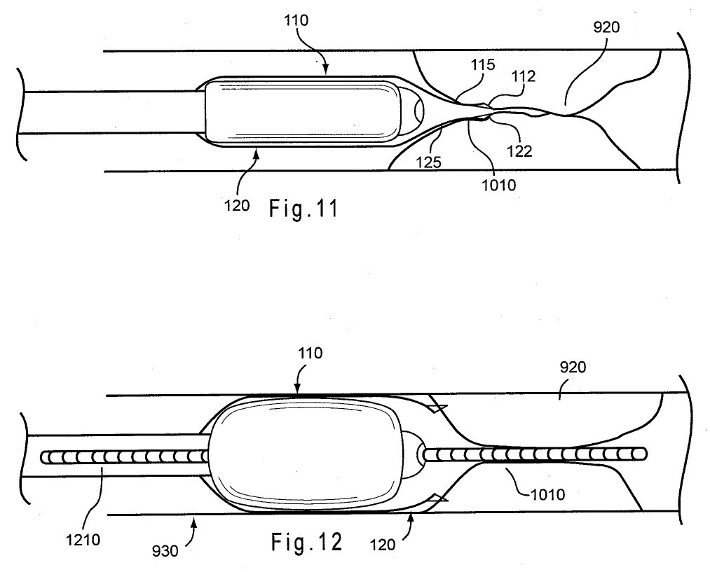

Having opened up the occlusion 920 to create a gap 1010, balloon 130 is re-deflated as shown in FIG. 11 to enable the dilator 100 to advance distally within the gap 1010 (FIG. 11). Specifically, the dilator 100 is advanced distally until the distal free ends 112 and 122 are inserted within gap 1010. FIG. 11 shows that more of the first strut 110 and the second strut 120 are in contact with occlusion 920. In particular, a second portion 115 of body portion 113 and a second portion 125 of body portion 120 are in contact with the occlusion 920. The penetration tip feature of the distal free ends 112 and 122 may be used to enhance engagement of the distal free ends 112 and 122 with the occlusion 920 so that the distal free ends 112 and 122 do not proximally slip out from gap 1010.

With the pre-clot dilator 100 configured as shown in FIG. 11, the balloon 130 is re-expanded to its inflated state. Movement of struts 110 and 120 along their respective first and second directions widens gap 1010 radially and longitudinally as shown in FIG. 12. Second portions 115 and 125 push against the occlusion 920 causing the occlusion 920 to be compressed towards the vessel wall 930. The gap 1010 is sufficiently widened to allow a guide wire 1210 to traverse the occlusion 920, as shown in FIG. 12. The guide wire 1210 is ultimately loaded through vessel 930 until it is within the target stenosed site at which angioplasty may occur. If advancement of the guide wire 1210 is impeded by a second occluded region, the pre-clot dilator 100 may be advanced to the second occluded region and the above-described procedure may be repeated for separating the second occlusion. This procedure may be continued until the guide wire 1210 reaches the target stenosed site. The balloon 930 remains sufficiently inflated so that struts 110 and 120 create sufficient clearance for guide wire 1210 to extend past the distal free ends 112 and 122, as shown in FIG. 12.

At this juncture, with the guide wire 1210 successfully deployed to its target site, the pre-clot dilator 100 may be withdrawn from the patient. The balloon 930 is re-deflated and struts 110 and 120 are configured so as to return to their retracted state, thereby creating a lower profile which can be removed through vessel 930 and the patient. After withdrawing the pre-clot dilator 100 from the patient, a conventional angioplasty balloon or cutting balloon may be loaded onto guide wire 1210 and advanced to the target site where angioplasty can be performed.

As can be seen, the pre-clot dilator 100 may be an effective tool for removing clotted regions located upstream of a target stenosed site. Conventional angioplasty balloons cannot remove such calcified lesions. Attempting to inflate a conventional angioplasty balloon and simultaneously advance it distally may simply rupture the balloon and/or cause the balloon to move in a proximal direction because of the hardness of the calcified lesion. Furthermore, cutting balloons as known in the art also are not capable of separating the clotted region because such balloons are designed to push out a stenosed region only when they are within the stenosed site. Removing the occlusion as described herein does not allow a device to be deployed within the stenosed site because the severity of the occlusion may completely block off the vessel passageway. As a result, the pre-clot dilator 100 as described is an effective tool for pushing out a stenosed region while being deployed upstream of the occlusion.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A pre-clot dilator, comprising:
a balloon having a distal portion, and a proximal portion, wherein at least a length of an outer surface of the balloon is expandable;
a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state;
at least one strut comprising a distal free end disposed past the distal end of the shaft, the strut proximally extending from the distal free end along the outer surface of the balloon and terminating at a proximal fixed end, the distal free end comprising a penetrating tip configured to pierce into an occlusion and upon expansion of the balloon move from a retracted state to an extended state away from the longitudinal axis of the shaft to spread apart the occlusion.

2. The pre-clot dilator of claim 1, wherein the distal free end moves away from the longitudinal axis of the shaft so as to pivotally radially extend away from the proximal fixed end of the at least one strut.

3. The pre-clot dilator of claim 1, wherein the proximal fixed end is attached to the outer surface of the balloon.

4. The pre-clot dilator of claim 1, wherein the proximal fixed end is attached to the shaft.

5. The pre-clot dilator of claim 1, wherein the penetrating tip comprises a sharpened edge sufficient to pierce into the calcified lesion.

6. The pre-clot dilator of claim 1, wherein an outer surface of the strut located proximal of the distal free end is non-sharpened.

7. The pre-clot dilator of claim 1, wherein the at least one strut comprises a first strut and a second strut, the first strut having a first distal free end and the second strut having a second distal free end, the first distal free end and the second distal free end converging towards each other in the retracted state.

8. The pre-clot dilator of claim 7, further comprising an elastic band disposed about the first strut, the second strut, and the balloon.

9. The pre-clot dilator of claim 7, further comprising an aspirator attached to the proximal end of the shaft.

10. The pre-clot dilator of claim 7, wherein the first distal free end comprises a first outer surface and the second distal free end comprises a second outer surface, the first outer surface and the second outer surface being sufficiently textured to frictionally engage the occlusion.

11. A pre-clot dilator, comprising:
a balloon having a distal portion, and a proximal portion, wherein at least a length of an outer surface of the balloon is expandable;
a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state;
a first strut comprising a first distal free end disposed past the distal end of the shaft, the first strut proximally extending from the from the first distal free end along the outer surface of the balloon and terminating at a first proximal fixed end attached to the distal end of the shaft;
a second strut spaced apart from the first strut, the second strut comprising a second distal free end disposed past the distal end of the shaft, the second strut proximally extending from the from the second distal free end along the outer surface of the balloon and terminating at a second proximal fixed end attached to the distal end of the shaft;
wherein the first distal free end comprises a first penetrating tip and the second distal free end comprises a second penetrating tip, the first and second penetrating tips pivotable from a retracted position to an extended state away from the longitudinal axis of the shaft.

12. The pre-clot dilator of claim 11, where the first proximal end and the second proximal end are attached to the distal end of the shaft by an adhesive or bond.

13. The pre-clot dilator of claim 11, wherein the first penetrating tip and the second penetrating tip converge towards each other.

14. The pre-clot dilator of claim 11, wherein the first penetrating tip and the second penetrating tip are spaced apart a first distance in a retracted position and further wherein the first penetrating tip and the second penetrating tip are spaced apart a second distance greater than the first distance in an expanded state.

15. A method for dilating an occluded region, comprising the steps of:
(a) providing a pre-clot dilator having
a balloon having a distal portion, and a proximal portion, wherein at least a length of an outer surface of the balloon is expandable;
a shaft having a longitudinal axis, a distal end and a proximal end, the balloon being mounted on the distal end of the shaft, wherein the shaft further comprises an inflation lumen extending therethrough in fluid communication with an interior region of the balloon, the balloon thereby being expandable between a deflated state and an inflated state;
at least one strut comprising a distal free end disposed past the distal end of the shaft, the strut proximally extending from the distal free end along the outer surface of the balloon and terminating at a proximal fixed end, the distal free end comprising a penetrating tip;
(b) advancing the pre-clot dilator proximal to the occluded region with the at least one strut collapsed along the outer surface of the balloon;
(c) piercing the occlusion with the penetrating tip;
(d) expanding the balloon to the inflated state; and
(e) pivotally expanding the at least one strut outward in a radial direction as the balloon is expanding to the inflated state to spread apart the occlusion.

16. The method of claim 15, further comprising the steps of:
(f) deflating the balloon;
(g) retracting the at least one strut to collapse the strut along the outer surface of the balloon; and
(h) advancing the pre-clot dilator further towards the occlusion.

17. The method of claim 16, further comprising the steps of:
(i) piercing the occlusion with the penetrating tip;
(j) re-inflating the balloon;
(k) expanding the balloon to the inflated state; and
(l) expanding the at least one strut outward as the balloon is expanding to the inflated state to further spread apart the occlusion to create a sufficient gap for a wire guide to advance therethrough

18. The method of claim 15, wherein the spreading apart of the occlusion creates a sufficient gap for a wire guide to advance therethrough to a target stenosed site.

19. The method of claim 18, further comprising the steps of:
(f) removing the pre-clot dilator from the wire guide;
(g) introducing a balloon catheter over the wire guide within the target stenosed site to perform angioplasty.

20. The method of claim 15, further comprising the step of:
(f) aspirating particles of the occlusion.
